(19) United States
(12) Patent Application Publication
(10) Pub. No.: US 2024/0170132 A1
(43) Pub. Date: May 23, 2024

(54) RADIOGRAPHING DEVICE

(71) Applicant: CANON KABUSHIKI KAISHA, Tokyo (JP)

(72) Inventor: HIROFUMI KUBOTA, Kanagawa (JP)

(21) Appl. No.: 18/511,856

(22) Filed: Nov. 16, 2023

(30) Foreign Application Priority Data
Nov. 21, 2022 (JP) 2022-185914

(51) Int. Cl. G16H 30/40

(52) U.S. Cl. CPC G16H 30/40

(57) ABSTRACT

A radiographing device includes an image acquisition unit that receives radiation that has passed through an examination target and acquires an image of the examination target, a storage unit that stores examination target information, a reading unit that reads identification information assigned to an examination target, a checking unit that checks the stored examination target information and the read identification information against each other, a notification unit that makes a notification of information about a result of the checking, and a storage unit that, based on the result of the checking, stores the image of the examination target and the read identification information in association with each other.

### BACKGROUND

### Field

The present disclosure relates to a radiographing device.

### Description of the Related Art

In recent years, radiographic imaging devices each incorporated in a stationary imaging system and portable flat panel detectors (hereinafter referred to as FPDs in some cases, and an FPD means a radiographing device) have been known. Because of the portability of a radiographing device (FPD), for example, the radiographing device is disposed near a patient in a radiographic room for imaging or is disposed outside a radiographic room for imaging at the time of the round of a patient who has difficulty in moving. There is a round-purpose radiographing device (FPD) that is used as a set with a control device for the radiographing device and a radiation generator for emitting radiation in synchronization with the radiographing device under the control of the control device.

The control device generally includes an input/output unit, an image storage unit, and a storage unit for storing examination information of each patient to be used at the time of rounds. The examination information is stored in the storage unit in advance before rounds, the round-purpose radiographing device is moved to the location of a patient, and then imaging is started after the examination information has been checked by the use of the input/output unit.

However, since the round-purpose radiographing device is large and heavy, there is a problem that it takes a lot of labor to move the radiographing device and the moving route of the radiographing device and the location where the radiographing device can perform imaging are limited.

As a method of overcoming the problem, a round method using only a radiographing device that automatically detects the emission of radiation and a radiation generator is considered. This round method not using a control device enables the reduction in the number of items to carry at the time of movement, size reduction and easier movement as compared with the round-purpose radiographing device, and the reduction in space required for imaging. However, since a storage unit for storing examination information is not present and an image is stored in the radiographing device, an image and examination information are separately managed. A problem therefore arises that a mix-up between an image and a patient occurs. PCT Japanese Translation Patent Publication No. 2018-525072 discloses a radiographing device that includes, for example, a bar code reader for reading patient identification information to prevent the occurrence of a mix-up between an image and a patient. Patient identification information is read before imaging and is added to an image to prevent the occurrence of a mix-up between an image and a patient.

However, in rounds in which, for example, imaging is performed upon a plurality of patients, the case may arise where a patient to be subjected to imaging is misidentified or the acquisition of patient identification information is forgotten. In this case, the radiographing device using the bar code reader disclosed in PCT Japanese Translation Patent Publication No. 2018-525072 cannot prevent the misidentification of a patient to be subjected to imaging.

### SUMMARY

The present disclosure provides a round-purpose radiographing device with which the misidentification of a patient to be subjected to imaging can be prevented. In a round, the radiographing device and a radiation generator are used. Patient examination information in the round is stored in the radiographing device. Read patient identification information and the patient examination information stored in the radiographing device are checked against each other. Based on a result of the checking, an alert is notified from the radiographing device to an operator.

A radiographing device according to an embodiment of the present disclosure includes an image acquisition unit configured to receive radiation that has passed through an examination target and acquire an image of the examination target, a storage unit configured to store examination target information, a reading unit configured to read identification information assigned to an examination target, a checking unit configured to check the stored examination target information and the read identification information against each other, a notification unit configured to make a notification of information about a result of the checking, and a storage unit configured to, based on the result of the checking, store the image of the examination target and the read identification information in association with each other.

Further features of the present disclosure will become apparent from the following description of exemplary embodiments with reference to the attached drawings.

### BRIEF DESCRIPTION OF THE DRAWINGS

FIG. 1 is a diagram illustrating the configuration of a radiographing system according to a first embodiment of the present disclosure.

FIG. 2 is a flowchart illustrating the process of a round operation performed by an operator described in the first embodiment of the present disclosure.

FIG. 3 is a conceptual diagram of a memory storing acquired patient examination information described in first embodiment of the present disclosure.

FIGS. 4A and 4B are flowcharts illustrating the operation process of a radiographing device (FPD) described in the first embodiment of the present disclosure.

FIG. 5 is a flowchart illustrating the process of a round operation performed by an operator described in a second embodiment of the present disclosure.

### DESCRIPTION OF THE EMBODIMENTS

The following embodiments describe X-rays applied as radiation, but radiation is not limited to X-rays and can be electromagnetic waves, a-rays, B-rays, or y-rays. The following embodiments describe a patient round being performed, but an embodiment of the present disclosure is applicable to the round examination of an examination target. In that sense, a patient in an embodiment can be read as an examination target.

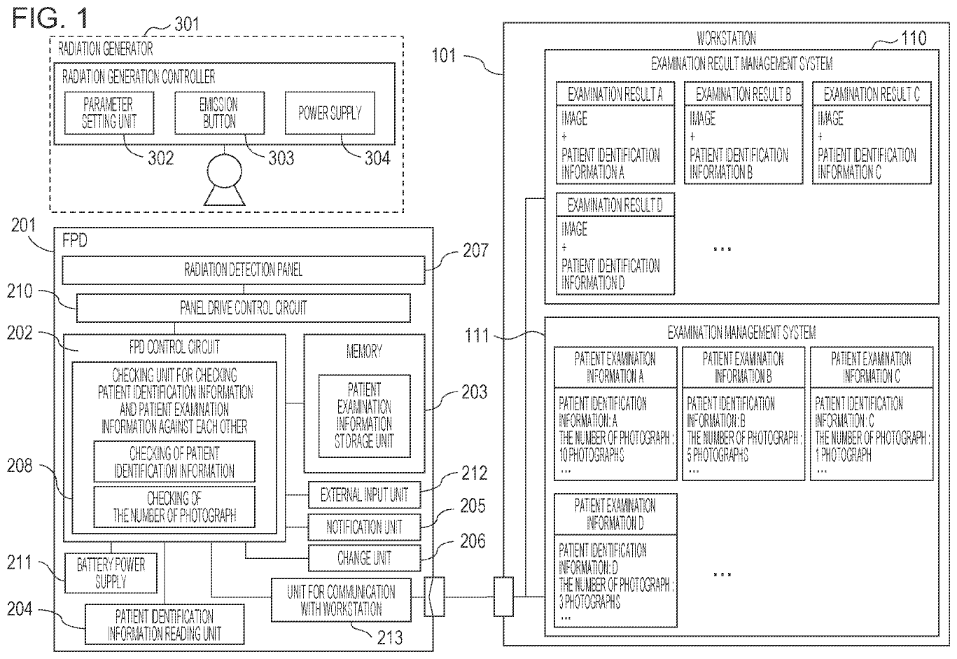

FIG. 1 is a diagram illustrating an example of a radiographing system according to a first embodiment of the present disclosure. This system is a medical radiographing system and includes a stationary workstation 101, a radiographing device (FPD) 201, and a radiation generator 301.

The radiation generator 301 emits X-rays to a subject or the radiographing device (FPD) 201. The radiation generator 301 according to the present embodiment can be moved to any location, includes a parameter setting unit 302, an emission button 303, and a power supply 304, and can emit X-rays out of synchronization with, for example, a radiographic imaging device. The parameter setting unit 302 sets parameters for, for example, a voltage, a current, and an emission time. When the emission button 303 is turned on, X-rays are emit based on settings input by the parameter setting unit 302.

The radiographing device (FPD) 201 includes a radiation detection panel 207, a panel drive control circuit 210, a battery power supply 211, a unit for communication with a workstation 213, and a memory 203. The radiographing device (FPD) 201 also includes a radiographing device control circuit (FPD control circuit) 202, a patient identification information reading unit 204, a notification unit 205, an external input unit 212, and a change unit 206.

The radiation detection panel 207 in the radiographing device (FPD) 201 functions as an image acquisition unit for acquiring the image of an examination target upon receiving radiation that has passed through a patient or an examination target. The radiation detection panel 207 and the panel drive control circuit 210 can store a signal charge based on radiation that has passed through a patient or an examination target and read a signal charge under the control of the FPD control circuit 202. The radiation detection panel 207 and the panel drive control circuit 210 can also convert a read signal charge from an analog value into digital data.

The FPD control circuit 202 controls an operation instruction for the panel drive control circuit 210. The FPD control circuit 202 stores in the memory 203 an image obtained by processing received digital data or patient examination information acquired from a workstation. The FPD control circuit 202 controls the overall operation of the radiographing device (FPD) 201, such as the management of a battery, communication with a workstation, and the operation of the patient identification information reading unit 204. The radiographing device (FPD) 201 includes the patient identification information reading unit 204 for reading identification information assigned to a patient or an examination target. Patient identification information read by the patient identification information reading unit 204 is checked whether it corresponds to examination information, which is information about a patient or an examination target, stored in a memory. It is also checked whether the number of photograph matches patient examination information. That is, the FPD control circuit 202 includes the checking unit 208 for checking information about an examination target stored in the memory 203 and identification information read by the patient identification information reading unit 204 against each other and sends an instruction to the notification unit 205 based on a result of the checking.

The memory 203 includes an image memory region functioning as a storage unit and a patient examination information memory region functioning as a patient examination information storage unit. In the image memory region, an image acquired by the image acquisition unit based on a checking result and identification information read by the patient identification information reading unit 204 are stored in association with each other. The radiographing device (FPD) control circuit 202 can read and write data from and in each region in the memory 203.

The radiographing device (FPD) 201 with the above configuration detects radiation emitted from the radiation generator 301 and passed through a subject and acquires a radiation image based on the subject.

The unit for communication with a workstation 213 is a unit for wired communication or near field communication. When the radiographing device (FPD) 201 and the workstation are connected in a wired manner, the connection portion between the radiographing device (FPD) 201 and a cable is a connector and they are detachable.

The notification unit 205 notifies a user of information about a result of checking of the checking unit 208 as a sound alert in response to an instruction from the FPD control circuit 202. For example, in the case where there is no match between identification information and information about an examination target as a result of checking of the checking unit 208, information about the result of the checking is notified as an alert. The notification unit 205 can use a display device, light, or vibrations instead of sound, and a plurality of notification units can be provided.

The change unit 206 can change information about a patient or an examination target stored in the patient examination information memory region. Specifically, a new patient or a new examination target can be added to information about a patient or an examination target stored in the patient examination information memory region.

The external input unit 212 transmits the response of an operator to the FPD control circuit 202. The external input unit 212 is not limited to a button and can be, for example, a touch panel.

The radiographing device (FPD) 201 includes a bar code reader as the patient identification information reading unit 204. A patient who is an examination target has a nameplate on the body thereof or a bed, and a bar code is printed on the nameplate. The FPD control circuit 202 can read a bar code on a nameplate using the patient identification information reading unit 204.

The patient identification information reading unit 204 is not limited to a bar code reader and can be a camera for reading, for example, a bar code or a camera for reading a numerical value or character information. In this case, patient identification information is managed in association with a code, a numerical value, or a character used by the patient identification information reading unit 204 and is assigned to a patient who is an examination target.

A bar code is provided on a bed in the present embodiment, but the method described in the present embodiment does not necessarily have to be employed. For example, a wristband or card with a bar code can be placed on a patient or provided at an entrance to a patient's room.

The radiographing device (FPD) 201 including the above elements can operate with power supplied from the battery power supply 211.

The workstation 101 is an information device disposed in a radiology department in a hospital. The workstation 101 includes an examination management system 111 and an examination result management system 110.

The examination management system 111 manages information about an examination performed on a patient in a round and accesses a radiology information system (RIS) or hospital information system (HIS) via an in-hospital network to acquire patient examination information.

Patient examination information includes the patient identification information of an examination target and information about imaging to be performed. The patient identification information includes one or more of a patient name, a patient ID, a date of birth, a gender, and a location. The information about imaging to be performed includes one or more of the date and time of an examination, an imaging procedure, the number of photograph, the amount of radiation exposure, and an examination order from among a plurality of patients or a plurality of examination targets. Patient examination information according to the present embodiment includes, for example, a patient ID and the number of photograph, but may include another piece of information.

The examination result management system 110 accesses a picture archiving and communication system (PACS) (image server) via an in-hospital network to manage an examination result such as an image output. The examination result management system manages an examination result transmitted from the radiographing device (FPD) 201 after a round. The examination result is information in which patient examination information used at the time of acquisition of a radiation image in an examination performed in a round is added.

Next, imaging performed using a radiographing system will be described focusing on a process performed by an operator.

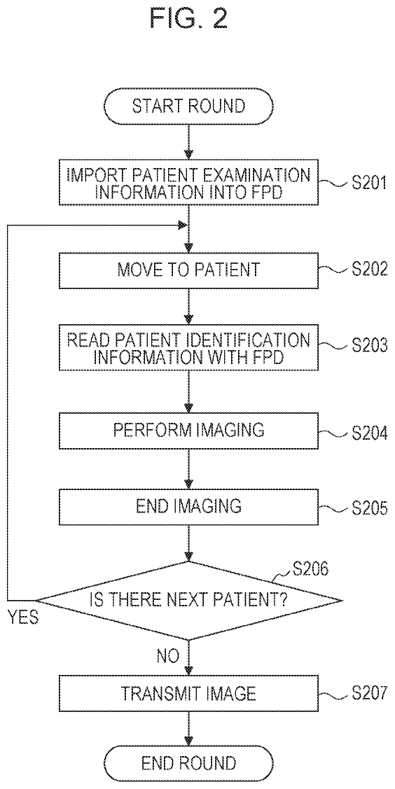

FIG. 2 is a flowchart illustrating the process of a round operation performed by the use of the radiographing device (FPD) 201.

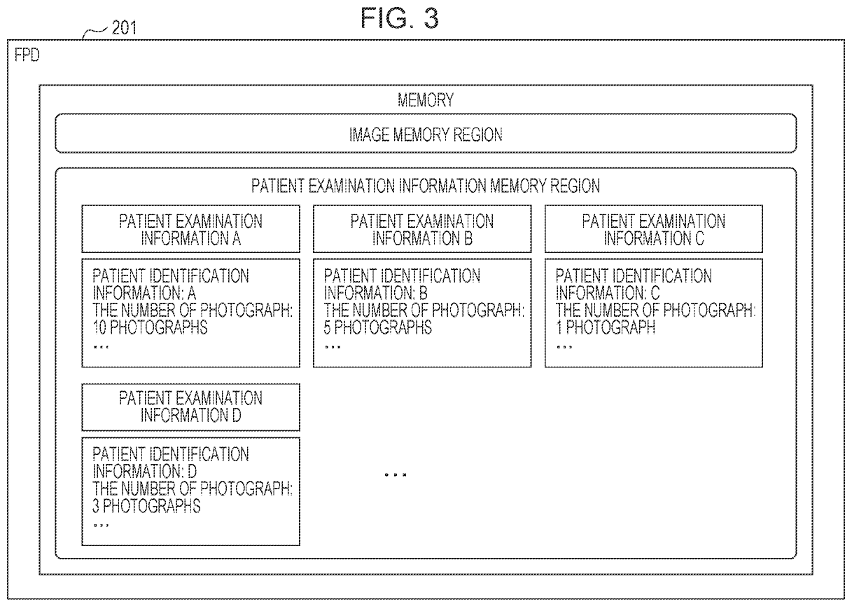

An operator connects the radiographing device (FPD) 201 to the workstation 101 to import patient examination information before visiting to a patient for rounds (S201). At that time, patient examination information is stored in the patient examination information memory region in the radiographing device (FPD) 201 (FIG. 3).

The pieces of patient examination information of a plurality of persons can be imported. After the import of patient examination information has been completed, the operator disconnects the radiographing device (FPD) 201 from the workstation 101 and moves to a patient with the radiographing device (FPD) 201 and the radiation generator 301 (S202).

After arriving at the patient, the operator activates the patient identification information reading unit 204 in the radiographing device (FPD) 201 using the external input unit 212 and reads patient identification information (S203). The patient is associated with a bar code corresponding to patient identification information. In the present embodiment, a bar code is provided on the bed of the patient who is an examination target. The radiographing device (FPD) 201 reads the bar code using the bar code reader included therein to recognize an imaging target. The operator checks the presence or absence of an alert 1 at this time and makes a response of, for example, YES or NO using the external input unit 212 when the alert 1 occurs. The alert 1 will be described below.

The operator then positions the radiographing device (FPD) 201 near the patient to acquire a radiation image (S204). The operator checks the presence or absence of an alert 2 at this time and makes a response of, for example, YES or NO using the external input unit 212 when the alert 2 occurs. The alert 2 will be described below. When the imaging of the required number of photograph is completed (S205), the operator moves to the next patient.

When the imaging related to all pieces of patient examination information registered in the radiographing device (FPD) 201 is completed (No in S206), the rounds end and the operator returns to the radiology department. The operator connects the radiographing device (FPD) 201 to the workstation 101 and transmits an examination result that is combined information of an image stored in the memory 203 and patient examination information to the examination result management system 110 (S207).

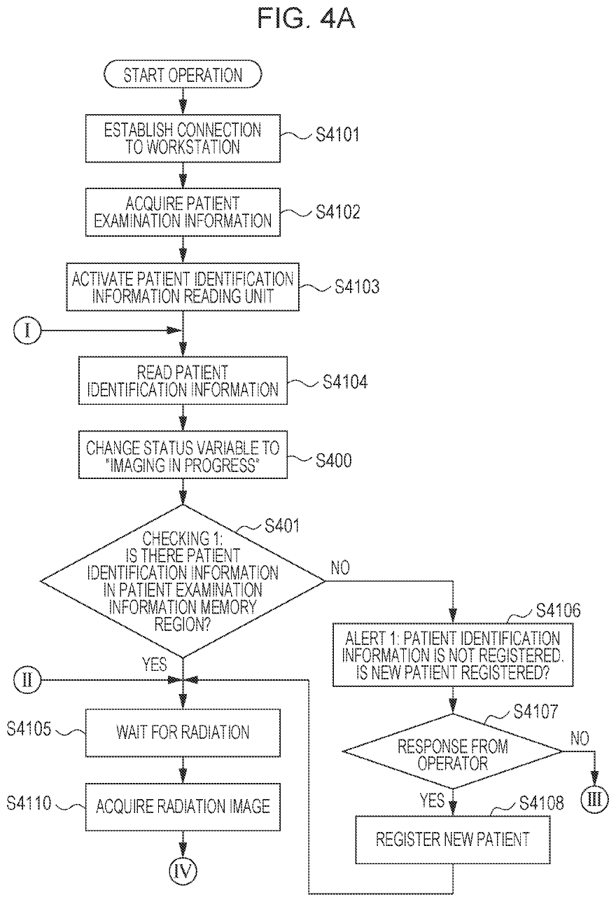

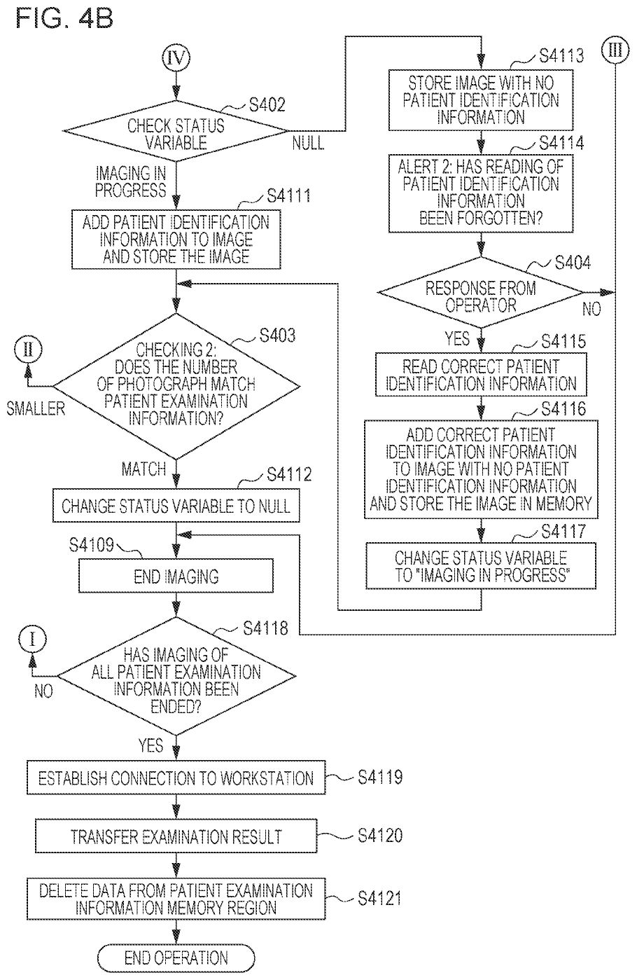

Next, imaging performed using the radiographing system will be described focusing on the process illustrated in FIGS. 4A and 4B performed by the radiographing device (FPD) 201. It is assumed that the radiographing device (FPD) 201 has a status variable. The status variable is “NULL” in a normal state, but is changed to “imaging in progress” in response to the reading of patient identification information. The expressions of the status variable, “imaging in progress” and “NULL”, are merely illustrative, and any expression can be optionally determined.

First, a preliminary preparation to perform the process illustrated in FIGS. 4A and 4B will be described. Before rounds using the radiographing device (FPD) 201, the patient examination information memory region in the memory 203 is empty. When an operator connects the radiographing device (FPD) 201 to the workstation 101 (S4101), the radiographing device (FPD) 201 receives patient examination information from the workstation 101 (S4102) and stores the patient examination information in the patient examination information memory region.

When the activated patient identification information reading unit 204 is held over a bar code corresponding to patient identification information (S4103), the radiographing device (FPD) 201 reads the bar code (S4104). A status variable is changed to “imaging in progress” in response to the reading of the bar code (S400). The radiographing device (FPD) 201 checks whether the read patient identification information is present in the patient examination information memory region based on the read patient identification information (S401). When there is corresponding patient identification information, the emission of radiation is waited for (S4105). When corresponding patient identification information is not present, the alert 1 is notified to an operator using the notification unit 205 (S4106). The alert 1 is that “Patient identification information is not registered. Is a new patient registered?”. The response to the alert 1 from the operator is waited for (S4107). When the response of Yes is received, a new patient is newly registered in the patient examination information memory region using the change unit 206 (S4108). When the response of No is received or there is no response, and imaging ends (S4109). When detecting the emission of radiation after waiting for the emission of radiation, the radiographing device (FPD) 201 sends an operation instruction to the panel drive control circuit 210 and acquires a radiation image (S4110).

When the acquisition of a radiation image ends, the radiographing device (FPD) 201 checks the status variable (S402). When the status variable is “imaging in progress”, patient identification information is added to the acquired radiation image and is stored in the image memory region (S4111). Then, the radiographing device (FPD) 201 checks whether the current number of photograph matches the number of photograph included in the patient examination information based on the read patient identification information (S403). When the current number of photograph is smaller, the emission of radiation is waited for and the acquisition of a radiation image is repeated. When the identification information of another patient or another examination target is read by the patient identification information reading unit 204 in the state where there is no match between the numbers of photograph, the notification unit 205 can make an alert notification. When the current number of photograph matches the number of photograph included in the patient examination information, the status variable is changed to “NULL” (S4112) and imaging ends (S4109). For the setting of the larger number of photograph is needed, the number of photograph included in patient examination information is changed using the change unit 206.

The alert 1 is notified by checking whether the read patient identification information is present in the patient examination information memory region based on the read patient identification information. This can prevent the occurrence of misidentification of a patient.

When the reading of patient identification information is not performed, S400 and S401 are skipped. Since S400 is skipped, the status variable remains “NULL”. The radiographing device (FPD) 201 proceeds to the acquisition of a radiation image and S402 in the state where the status variable remains “NULL”. When the status variable is “NULL”, an acquired radiation image is stored in the image memory region as an image with no patient identification information (S4113) and the radiographing device (FPD) 201 notifies the operator of an alert 2 using the notification unit 205 (S4114). The alert 2 is that “Has reading of patient identification information been forgotten?”. When the response of Yes to the alert 2 is received from the operator, the operator is prompted to read correct patient identification information. When correct patient identification information is input, the patient identification information is added to the image with no patient identification information and is stored in the image memory region (S4116). The status variable is changed to “imaging in progress” (S4117) and the process proceeds to S403.

The status variable is changed by checking whether the number of photograph matches patient examination information. The notification of the alert 2 is made based on the status variable, and this can prevent the non-performance of reading of patient identification information. The checking of whether the number of photograph matches patient examination information can be performed by checking the number of images associated with identification information stored in the image memory region. That is, the checking unit 208 can check the number of photograph included in examination information against the number of images that is stored in association with identification information including information matching information about an examination target including the number of photograph. When this checking results in no matching, the notification unit 205 makes a notification of information about a result of the checking as an alert.

When the number of photograph as patient examination information, that is, information about an examination target, is empty, information of the number of photograph can be handled as one or the number of photograph (the standard number of photograph) determined in advance. That is, when imaging starts without the reading of patient identification information that is information about an examination target, there is no match resulting from the checking of the number of photograph and an alert is notified because there is no image stored in association with identification information corresponding to this examination target. Since the notification unit 205 makes the notification of an alert for imaging performed without the reading of identification information, the non-performance of reading of identification information can be prevented at the time of imaging. When the patient identification information reading unit 204 reads identification information after the alert has been notified, the read identification information can be stored in association with an image with which identification information has not been associated because of the non-performance of reading of identification information.

The radiographing device (FPD) 201 can change the number of photograph in patient examination information as appropriate using the change unit 206.

When a round order is determined, the round order can also be included in patient examination information and stored in the patient examination information memory region. When the reading of patient identification information is not performed in the determined order, an alert can be notified. That is, when the imaging of a plurality of patients or a plurality of examination targets is performed, information about an examination target includes the examination order from among the multiple examination targets. In this case, the checking unit 208 can check the order where the patient identification information reading unit 204 reads identification information and the examination order from among a plurality of examination targets against each other. When there is no match resulting from the checking, the notification unit 205 can make the notification of information about the checking result.

The radiographing device (FPD) 201 includes the patient identification information reading unit 204 in the present embodiment, but the radiographing device (FPD) 201 does not necessarily have to include the patient identification information reading unit 204. For example, a peripheral device such as a round-purpose cart included in the configuration of a round-purpose radiographing device can include the patient identification information reading unit 204.

When the imaging related to all pieces of information in the patient examination information memory region ends (YES in S4118), the radiographing device (FPD) 201 waits for transferring results. When the radiographing device (FPD) 201 is connected to the workstation 101 (S4119), the radiographing device (FPD) 201 transfers data in the image memory region to the examination result management system 110 in the workstation 101 (S4120). At the same time, the radiographing device (FPD) 201 deletes data from the patient examination information memory region (S4121) and waits for receiving new patient examination information.

In the present embodiment, the occurrence of misidentification of an imaging target patient or the occurrence of misidentification of a patient due to, for example, the non-performance of reading of patient identification information can be prevented by causing the radiographing device (FPD) 201 to check patient identification information and patient examination information against each other as illustrated in FIGS. 4A and 4B.

A radiographing system according to a second embodiment has the same configuration as the radiographing system according to the first embodiment. Respective constituent elements are denoted by the same reference numerals and the description(s) thereof will be omitted.

A round process and a preparation process in the present embodiment are the same as those performed in the radiation system according to the first embodiment. Respective elements are denoted by the same reference numerals and the description(s) thereof will be omitted (S201 to S207).

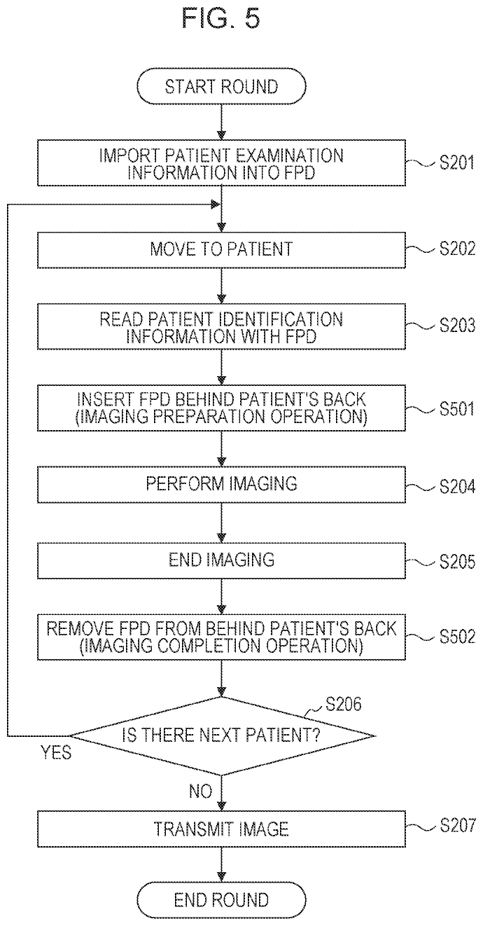

FIG. 5 illustrates the process of a round operation performed by an operator according to the present embodiment in more detail.

Before and after radiation imaging, there are timings when the radiographing device (FPD) 201 is inserted behind a patient's back (S501) and then removed from behind the patient's back (S502). That is, there are operations for preparing for and completing (ending) imaging. When any detection unit for detecting these operations detects the imaging preparation operation or the imaging completion operation at these timings, the notification unit 205 makes the notification of the alert 2. Examples of a detection unit include an acceleration sensor, a shock sensor, a gravity sensor, and a vibration sensor.

In the case where the reading of patient identification information is not performed (the status variable is “NULL”) when it is detected that the radiographing device (FPD) 201 has been inserted behind a patient's back, the notification unit 205 notifies an operator of the alert 2.

When the response of Yes is received from the operator, the radiographing device (FPD) 201 prompts the operator to read patient identification information and waits for the input of patient identification information. The process after patient identification information has been input is the same as that in the first embodiment described above.

In the case where the reading of patient identification information is not performed (the status variable is “NULL”) when it is detected that the radiographing device (FPD) 201 has been removed from behind the patient's back, the radiographing device (FPD) 201 notifies an operator of the alert 2 using the notification unit 205.

Thus, when the detection unit detects the imaging preparation operation or the imaging completion operation in the state where the patient identification information reading unit 204 does not complete the reading of identification information, the notification unit 205 can determine that there is no match resulting from checking and make the notification of information about the checking result. The process after the notification of the alert 2 has been made is the same as that in the first embodiment described above.

By increasing the number of timings when an alert is generated as compared with the first embodiment, the occurrence of misidentification of a patient can be even more effectively prevented.

According to the present disclosure, the occurrence of misidentification of an imaging target patient or the occurrence of misidentification of a patient due to, for example, the non-performance of acquisition of patient identification information can be prevented by causing a radiographing device to check patient identification information and patient examination information against each other.

### Other Embodiments

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2022-185914 filed Nov. 21, 2022, which is hereby incorporated by reference herein in its entirety.

What is claimed is:

1. A radiographing device comprising:
an image acquisition unit configured to receive radiation that has passed through an examination target and acquire an image of the examination target;
a storing unit configured to store examination target information;
a reading unit configured to read identification information assigned to an examination target;
a checking unit configured to check the stored examination target information and the read identification information against each other;
a notification unit configured to make a notification of information about a result of the checking; and
a storage unit configured to, based on the result of the checking, store the image of the examination target and the read identification information in association with each other.

2. The radiographing device according to claim 1, wherein the identification information includes one or more of a patient name, a patient ID, a date of birth, a gender, or a location.

3. The radiographing device according to claim 1, wherein the notification unit makes a notification of the information about a result of checking via a sound or via light.

4. The radiographing device according to claim 1, further comprising a detection unit configured to detect imaging preparation or imaging completion,
wherein, when the detection unit detects imaging preparation or imaging completion in a state where the reading unit has not completed reading of identification information, the notification unit determines that there is no match as the result of checking and makes a notification of information about the result of the checking.

5. The radiographing device according to claim 1, wherein, when there is no match between the read identification information and the stored examination target information as a result of the checking, the notification unit makes a notification of information about the result of the checking.

6. The radiographing device according to claim 5, further comprising a change unit configured to change the stored examination target information.

7. The radiographing device according to claim 6, wherein the change unit is capable of adding an examination target to the stored examination target information.

8. The radiographing device according to claim 1, wherein the stored examination target information includes one or more of a patient name, a patient ID, a date of birth, a gender, or a location and one or more of a date and time of an examination, an imaging procedure, the number of photograph, the amount of radiation exposure, or an examination order from among a plurality of examination targets.

9. The radiographing device according to claim 8,
wherein the stored examination target information includes an examination order from among a plurality of examination targets,
wherein the checking unit is capable of checking an order where the reading unit reads identification information and the examination order from among a plurality of examination targets against each other, and
wherein, when there is no match as a result of the checking, the notification unit makes a notification of information about the result of the checking.

10. The radiographing device according to claim 8,
wherein the stored examination target information includes the number of photograph,
wherein the checking unit is capable of checking the number of photograph and a number of images stored in association with identification information including information matching the stored examination target information including the number of photograph against each other, and
wherein, when the reading unit reads new identification information in a state where there is no match as a result of the checking, the notification unit makes a notification of information about the result of the checking.

11. The radiographing device according to claim 8,
wherein the stored examination target information includes the number of photograph,
wherein the checking unit is capable of checking the number of photograph and a number of images stored in association with the read identification information including information matching the stored examination target information including the number of photograph against each other, and
wherein, when there is no match as a result of the checking, the notification unit makes a notification of information about the result of the checking.

12. The radiographing device according to claim 11, wherein, when the reading unit reads identification information after the notification unit has made a notification, an image not associated with identification information is associated with the read identification information and stored in the storage unit.
